(19) United States
(12) Patent Application Publication
(10) Pub. No.: US 2026/0294371 A1
(43) Pub. Date: Oct. 1, 2026

(54) RADIOGRAPHIC IMAGING SYSTEM, INFORMATION PROCESSING DEVICE AND METHOD OF CONTROLLING SAME, AND PROGRAM

(71) Applicant: CANON KABUSHIKI KAISHA, Tokyo (JP)

(72) Inventors: HIKARU TANAKA, Kanagawa (JP); KAZUMA OBARA, Tokyo (JP); YUICHI NISHII, Kanagawa (JP)

(21) Appl. No.: 19/704,248

(22) Filed: Jun. 10, 2026

Related U.S. Application Data
(63) Continuation of application No. 18/889,045, filed on Sep. 18, 2024.

(30) Foreign Application Priority Data
Sep. 29, 2023 (JP) 2023-169954
Sep. 29, 2023 (JP) 2023-169955
Oct. 2, 2023 (JP) 2023-171699
Feb. 22, 2024 (JP) 2024-025120

(51) Int. Cl. A61B 6/46; A61B 6/00; A61B 6/04

(52) U.S. Cl. CPC A61B 6/463; A61B 6/04; A61B 6/461; A61B 6/465; A61B 6/5294

(57) ABSTRACT

A radiographic imaging device includes an optical image capture device that captures an optical image of a subject of radiography; and an information processing device that causes a display unit to display the optical image acquired by the optical image capture device. The information processing device acquires information on a step according to progress from among a plurality of steps related to the radiography. The information processing device stops the display of the optical image on the display unit on the basis of the step indicated by the information being a predetermined step among the plurality of steps.

### CROSS-REFERENCE TO RELATED APPLICATIONS

This application is a continuation of co-pending U.S. patent application Ser. No. 18/889,045, filed Sep. 18, 2024, which claims priority to Japanese Patent Application Nos. 2023-169955, filed Sep. 29, 2023, 2023-169954, filed Sep. 29, 2023, 2023-171699, filed Oct. 2, 2023, and 2024-025120 filed Feb. 22, 2024, all of which are hereby incorporated by reference herein in their entireties.

### BACKGROUND

### Field

The present disclosure relates to a radiographic imaging system that uses optical images. This radiographic imaging system includes an information processing device that displays information pertaining to radiographic imaging using a radiographic imaging device, and software to be executed by the information processing device. The radiographic imaging device is a flat panel detector (FPD), for example. The information processing device is a PC, for example. The software is an imaging management application, for example.

### Description of the Related Art

Radiographic imaging devices using a flat panel detector (FPD) formed by semiconductor materials are widespread as imaging devices used for radiation-based diagnostic medical imaging and nondestructive testing. In diagnostic medical imaging, for example, such radiographic imaging devices are used as digital imaging devices for still imaging, such as general radiography, and dynamic imaging, such as fluoroscopy.

In radiography, subject positioning is performed as a preliminary step, and it has been observed that in some cases, improper positioning can result in rejection. Recently, a radiographic imaging system (radiography system) provided with an optical camera (optical image capture device) that allows for checking of the status of the subject has been proposed as a configuration to assist with positioning.

Japanese Patent Laid-Open No. 2013-48740 describes means for guiding subject positioning suitable for radiography by using a subject image acquired by an optical image capture device during radiography. Also, in Japanese Unexamined Patent Application Publication No. 2020-199163, an image acquired from an optical image capture device can be analyzed, and an analysis result can be displayed on a screen for radiography to notify a user.

The systems described in Japanese Patent Laid-Open No. 2013-48740 and No. 2020-199163 have room for improvement from an ease-of-use standpoint. It is desirable that an image or information based thereon acquired from an optical image capture device in this way is outputted with content and at timings suited to the steps of the radiography process.

### SUMMARY

The present disclosure was devised in light of the issues described above, and an objective thereof is to provide a radiographic imaging system in which output information obtained on the basis of an optical image capture device can be outputted appropriately to suit the steps of the radiography process.

The present disclosure provides a radiographic imaging device that includes an optical image capture device that captures an optical image of a subject of radiography, and an information processing device that causes a display unit to display the optical image acquired by the optical image capture device, wherein the information processing device acquires information on a step according to progress from among a plurality of steps related to the radiography, and wherein the information processing device stops the display of the optical image on the display unit on the basis of the step indicated by the information being a predetermined step among the plurality of steps.

Further features of the present disclosure will become apparent from the following description of exemplary embodiments with reference to the attached drawings.

### BRIEF DESCRIPTION OF THE DRAWINGS

FIG. 1 is a diagram illustrating a configuration of a radiographic imaging system.

FIG. 2 is a diagram illustrating functional blocks of a control device.

FIG. 3 is a diagram illustrating a hardware configuration of a control device.

FIG. 4 is a diagram illustrating a use sequence of a camera coordination system.

FIG. 5 is a diagram illustrating an optical image display example in an imaging management application.

FIG. 6 is a diagram illustrating an example of a display screen immediately after radiography in an imaging management application.

FIG. 7 is a diagram illustrating an example of a display screen during imaging standby in an imaging management application.

FIG. 8 is a diagram illustrating a flowchart of optical image display.

FIG. 9 is a diagram illustrating an optical image display example in an imaging management application.

FIG. 10 is a diagram illustrating an optical image display example in an imaging management application.

FIG. 11 is a diagram illustrating a flowchart of optical image display.

FIG. 12A is a diagram illustrating a situation in which the display of an optical image is turned off and FIG. 12B is a diagram illustrating a situation in which image processing is applied to an optical image.

FIG. 13 is a diagram illustrating a flowchart of optical image display.

FIG. 14A is a diagram illustrating an analysis result notification example 1 in an imaging management application and FIG. 14B is a diagram illustrating an analysis result notification example 2 in an imaging management application.

FIG. 15 is a diagram illustrating a flowchart of an examination process.

FIG. 16 is a diagram illustrating a flowchart of an optical image analysis process.

FIG. 17 is a diagram illustrating the correspondence between determination results from image analysis and detection result notifications.

FIG. 18 is a diagram illustrating an optical image display example 2 in an imaging management application.

FIG. 19 is a diagram illustrating a radiography method settings screen in an imaging management application.

FIG. 20A is a diagram illustrating a table of radiography information management method (a), FIG. 20B is a diagram illustrating a table of radiography information management method (b), FIG. 20C is a diagram illustrating a table of radiography information management method (c), FIG. 20D is a diagram illustrating a table of radiography information management method (d), and FIG. 20E is a diagram illustrating a table of radiography information management method (e).

FIG. 21 is a diagram illustrating a flowchart of radiography information management method (a).

FIG. 22 is a diagram illustrating a flowchart of radiography information management method (b).

FIG. 23 is a diagram illustrating a flowchart of radiography information management method (c).

FIG. 24 is a diagram illustrating a flowchart of radiography information management method (d).

FIG. 25 is a diagram illustrating a flowchart of radiography information management method (e).

### DESCRIPTION OF THE EMBODIMENTS

Hereinafter, embodiments of the present disclosure will be described in detail using the drawings. Note that the disclosure as recited in the claims is not limited to the configurations described in the embodiments. Portions of configurations and portions of processes may be replaced with equivalents, omitted, or otherwise modified insofar as similar effects are obtained.

### First embodiment

### Radiographic Imaging System

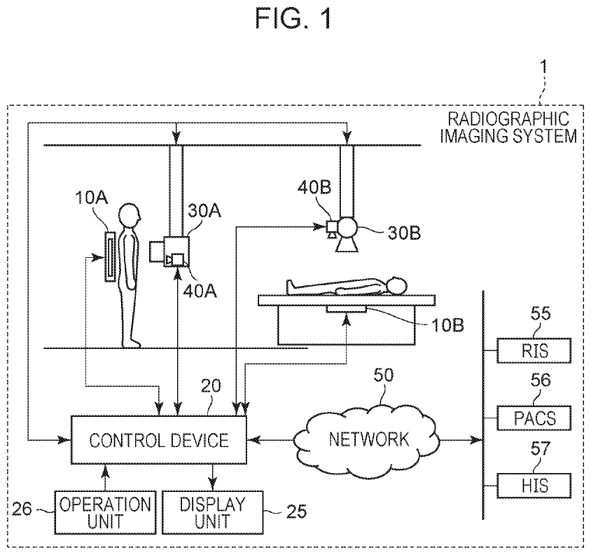

The following describes a radiographic imaging system 1, which is the usage environment of a radiation detecting device 10 (radiographic device, radiographic imaging device). FIG. 1 is a diagram illustrating a schematic configuration of a radiographic imaging system.

The radiographic imaging system 1 includes a radiographic detecting device 10 (10A, 10B), a control device 20, a radiation generating device 30 (30A, 30B), an optical image capture device 40 (40A, 40B), a display unit 25, an operation unit 26, an RIS 55, a PACS 56, and an HIS 57.

The control device 20 is an information processing device that acts as a relay for the radiation detecting device 10, the radiation generating device 30, and each device connectible over a network 50, and carries out various types of control. The details of the control device 20 will be described later.

The radiation generating device 30 (radiation emitting device) is provided with a radiation tube that generates radiation, and irradiates a patient or other subject with radiation. Herein, the radiation is assumed to include not only X-rays but also alpha rays, beta rays, gamma rays, particle rays, cosmic rays, and the like. It is assumed that a suitable device out of a radiation generating device 30A and a radiation generating device 30B is selected and used according to the radiography details.

Unless specifically noted otherwise, these are collectively referred to as the radiation generating device 30.

The radiation detecting device 10 (radiographic device, radiographic imaging device) is a device that generates an image on the basis of radiation emitted from the radiation generating device 30. The radiation detecting device 10 is a flat panel detector, for example. It is assumed that a suitable device out of a radiation detecting device 10A and a radiation detecting device 10B is selected and used according to the radiography details. Unless specifically noted otherwise, these are collectively referred to as the radiation detecting device 10.

The radiation detecting device 10 detects radiation that has been emitted from the radiation generating device 30 and passed through the examinee (subject). Note that image data may also be referred to as a medical image or a radiograph. Specifically, the radiation detecting device 10 detects radiation that has passed through the examinee as electric charge corresponding to the transmitted radiation dose. For example, a direct-conversion-type sensor that directly converts radiation into electric charge, such as a-Se, which converts radiation into electric charge, or an indirect-type sensor that uses a scintillator such as CsI, which converts radiation into visible light, and a photoelectric conversion element such as a-Si, may be used in the radiation detecting device 10. Furthermore, the radiation detecting device 10 generates image data by A/D converting the detected electric charge, and outputs the generated image data to the control device 20.

The optical image capture device 40 is a camera device for capturing the status of the examinee undergoing radiography. It is assumed that a suitable device out of an optical image capture device 40A and an optical image capture device 40B is selected and used according to the radiography details. Unless specifically noted otherwise, these are collectively referred to as the optical image capture device 40.

The display unit 25 is a display device which is provided with a monitor, such as a liquid crystal display, and which is capable of displaying information. The operation unit 26 is an input device provided with a keyboard, a pointing device (such as a mouse, for example), a touch panel, or the like.

The RIS 55, the PACS 56, and the HIS 57 are services that cooperate with the control device 20 over a network to extend various functions related to radiography. In addition, the control device 20 is connected to the RIS 55, the PACS 56, and the HIS 57 over the network 50, and can exchange radiographs, patient information, and the like. FIG. 1 illustrates the RIS 55, the PACS 56, and the HIS 57 as being included in the radiographic imaging system 1, but a system that does not include at least one of the above is also possible. Herein, RIS is an abbreviation of Radiology Information Systems. PACS is an abbreviation of Picture Archiving and Communication Systems. HIS is an abbreviation of Hospital Information Systems.

The control device 20 controls radiography using the radiation detecting device 10 and the radiation generating device 30.

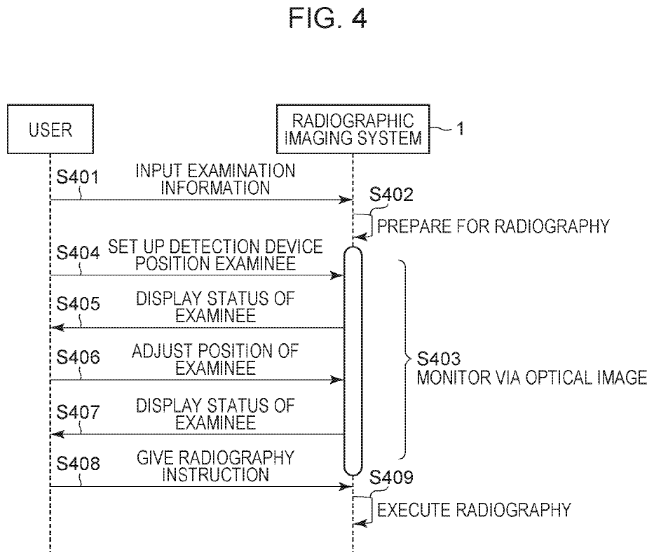

The radiographic imaging device according to the present embodiment is used according to a sequence like the one illustrated in FIG. 4, for example.

In step 401 (hereinafter denoted S401 and so on), a user (radiologist) who uses the radiographic imaging system inputs examination information into the radiographic imaging system 1.

In S402, the radiographic imaging system 1 starts preparing for radiography.

In S403, the radiographic imaging system 1 monitors the examinee by optical image capture as one of the processes during the radiography preparation period.

In S404, the user arranges the radiation detecting device 10 and the examinee.

In S405, the user checks the status of the examinee displayed in the radiographic imaging system 1.

In S406, if the positional relationship of the radiation detecting device 10 and the examinee is not appropriate, the user adjusts the positions of the radiation detecting device 10 and the examinee.

In S407, the user checks the status of the examinee displayed in the radiographic imaging system 1.

In S408, the user confirms that the positional relationship of the radiation detecting device 10 and the examinee is appropriate, and instructs the radiographic imaging system to execute radiography.

In S409, the radiographic imaging system 1 executes radiography.

### Control Device

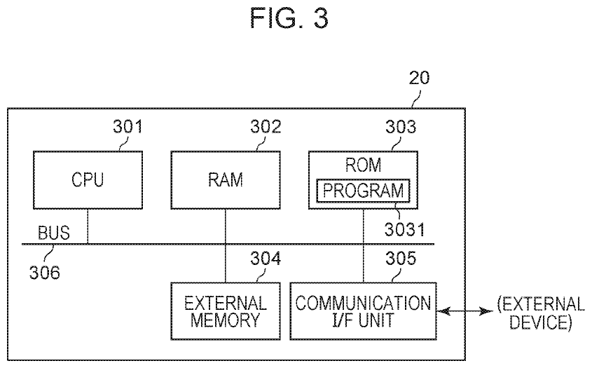

FIG. 3 is a diagram illustrating a configuration of a control device. The control device 20 includes a CPU 301, RAM 302, ROM 303, external memory 304, a communication I/F unit 305, and a bus 306. The CPU 301, RAM 302, ROM 303, external memory 304, and communication I/F unit 305 are communicatively interconnected via the bus 306.

The CPU 301 (central processing unit) centrally controls operations by the control device 20 and controls each of the components illustrated in FIG. 3 via the bus 306.

The RAM 302 (writable memory) functions as the main memory, work area, and the like of the CPU 301. When executing processing, the CPU 301 loads a required computer program 3031, data, and the like from the ROM 303 into the RAM 302 and executes the computer program 3031 and the like to thereby realize various functional operations. The control device 20 has an application function that runs on a computer. That is, the control device 20 has at least one processor and a memory, and realizes the functional units described below by causing the processor to execute a program stored in the memory. However, some or all of the functional units may also be realized by special-purpose hardware.

The ROM 303 (read-only memory) stores the computer program 3031, data, and the like required for the CPU 301 to execute processing. Note that the computer program 3031, data, and the like may also be stored in the external memory 304.

The external memory 304 is a mass storage device, and is realized by a hard disk device, IC memory, or the like. For example, the external memory 304 stores various data, various information, and the like required when the CPU 301 executes the computer program 3031 and the like to perform processing. Also, for example, the external memory 304 stores various data, various information, and the like obtained as a result of the CPU 301 executing the computer program 3031 and the like to perform processing.

The communication I/F (interface) unit 305 is responsible for communication between the control device 20 and external equipment. Through the communication I/F unit 305, the control device 20 is connected to the radiation generating device 30, the radiation detecting device 10, and the optical image capture device 40 over a wired or wireless network, or on a dedicated line.

The bus 306 is for communicatively connecting the CPU 301 with the RAM 302, ROM 303, external memory 304, and communication I/F unit 305.

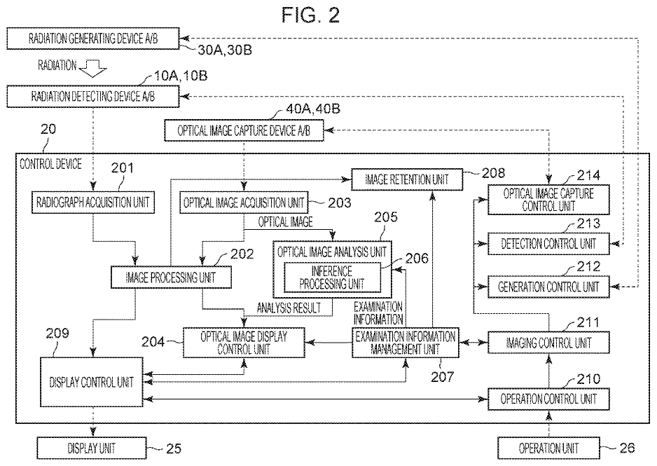

FIG. 2 is a diagram illustrating functional blocks of a control device.

An imaging control unit 211 of the control device 20 controls a generation control unit 212, a detection control unit 213, and an optical image capture control unit 214 on the basis of an instruction from an operation control unit 210 and examination information managed by an examination information management unit. The examination information includes information such as the dose, irradiation time (ms), tube current (mA), tube voltage (kV), and the receptor field, which is the area where radiation is detected. This information is transmitted to the radiation detecting device 10 via the image capture control unit 211 and the detection control unit 213.

The generation control unit 212 controls the timing of radiation generation by the radiation generating device 30 and the radiography conditions.

The generation control unit 212 outputs information such as an irradiation control signal to the radiation generating device 30 on the basis of the dose information and the like. The irradiation control signal conveyed from the generation control unit 212 to the radiation generating device 30 may include two signals, namely a stop signal to stop emitting radiation (irradiation stop signal) and an irradiate signal to emit radiation (non-irradiation stop signal). By controlling the output of one or both of the stop signal and the irradiate signal, the generation control unit 212 can control the emitting and stopping of radiation from the radiation generating device 30.

The detection control unit 213 communicates with the radiation detecting device 10 to carry out various types of control for radiographic imaging (radiography). For example, the detection control unit 213 executes various types of communication processing associated with radiographic imaging with respect to the radiation detecting device 10. In this communication processing, imaging condition settings information, operation control settings information, image information, entrance surface dose information, and the like are exchanged.

The optical image capture control unit 214 controls the start and end of image capture by the optical image capture device 40, conditions for acquiring optical images, timing, frame rate, zoom, focus, and other functions.

A radiograph acquisition unit 201 receives image data from the radiation detecting device 10. An image processing unit 202 performs image processing on the received image data, as necessary, and provides the processed image data to a display control unit 209 and an image storage unit 208.

The display control unit 209 provides, to the display unit 25, screen information for an imaging management application that enables viewing of information about radiography. A graphical user interface of the imaging management application displayed on the display unit 25 is linked to operations performed using the operation unit 26. Consequently, information related to the imaging management application can be entered via the operation unit 26. For example, in the case where multiple pieces of examination information are displayed on the display unit 25 in the form of a list, the operation unit 26 can be used to provide operating input to select one piece of examination information from the list. The selected examination information can be set as an examination target. Note that the user may also enter examination information directly from the operation unit 26. Examination information entered on the imaging management application is managed by an examination information management unit 207.

An optical image acquisition unit 203 acquires an optical image from the optical image capture device 40, and provides the acquired optical image to the image processing unit 202 and an optical image analysis unit 205. The optical image acquisition unit 203 is capable of acquiring an optical image from one or both of the optical image capture devices 40A, 40B. The optical image is preferably a dynamic image, but may also be a still image acquired sporadically. The image processing unit 202 performs image processing on the received image data, as necessary, and provides the processed image data to an optical image display control unit 204 and the image storage unit 208. In addition, the optical image to be used for analysis may also be an optical image with a decimated frame rate lower than the acquisition frame rate of the optical image capture device 40.

The optical image analysis unit 205 makes a determination using the optical image acquired from the optical image acquisition unit 203 and the examination information acquired from the examination information management unit 207. Specifically, the position, type, and posture of the area of the human body of the subject in the optical image are analyzed to determine if the above match the settings in the examination information. In the present embodiment, an inference processing unit 206 using machine learning is used in the determination by the optical image analysis unit 205, but the determination may also be made according to another method.

The optical image display control unit 204 controls display related to the image obtained by the optical image capture device 40. The optical image display control unit 204 alters and adds information to the optical image, for example, and provides the result as image information to the display control unit 209. The image information provided to the display control unit 209 is used as part of the information to be displayed on the graphical user interface of the imaging management application, and is displayed on the display unit 25.

Note that in FIG. 1, the control device 20 is illustrated as a single device to facilitate explanation, but the control device 20 may also be formed from multiple devices. For example, the optical image capture control unit 214, the detection control unit 213, the generation control unit 212, and the display control unit 209 may also be respectively independent control devices, and may also be a combination of control devices selectively provided with multiple functions from among these units.

### Display of Optical Image

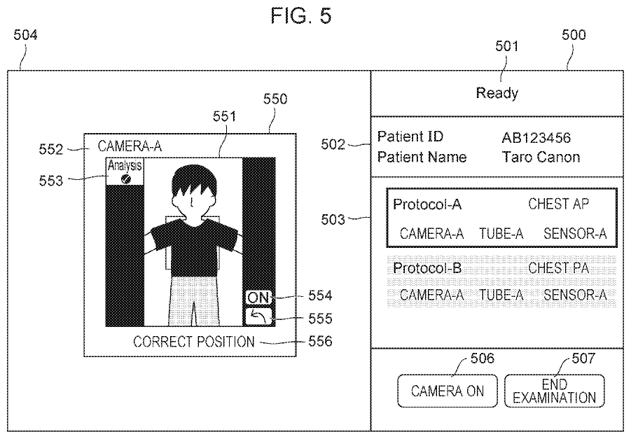

Next, FIG. 5 will be used to describe the display of the optical image. FIG. 5 is a diagram illustrating an optical image display example in an imaging management application.

In the present embodiment, the status of the examinee during radiography is captured in an image by the optical image capture device 40, and the optical image obtained thereby is used for status monitoring.

At the start of a radiographic examination, a screen 500 is displayed on the GUI of the imaging management application.

The screen 500 includes a radiography status 501, examinee information 502, examination information 503, a radiograph display area 504, an optical image window 550, an optical image window display toggle button 506, and an end examination button 507.

The radiography status 501 is information indicating the availability (suitability) of starting radiography (imaging operation). In the present embodiment, radiography is started with a start radiography button (not illustrated), but the radiography status 501 may also double as the start radiography button. The examinee information 502 is information about the examinee who is to undergo radiography.

The examination information 503 is information such as radiograph details for the current examination and a thumbnail (reduced image) of a radiograph acquired by the radiograph acquisition unit 201. The user can select one set of radiography details from among the radiography details listed in the examination information 503 to prepare for the next instance of radiography. By selecting a set of radiography details, the radiation detecting device 10 is instructed to prepare for radiography. This allows one of multiple steps for performing radiography to progress. On the screen 500, the “Protocol-A” radiography details are in the selected state.

The radiograph display area 504 is an area for displaying a radiograph acquired by the radiograph acquisition unit 201 in the current examination. At the stage of displaying the screen 500, radiography has not been performed yet, and thus the radiograph display area 504 is blank, with no radiograph placed therein. Information such as a date and time pertaining to the current radiography session may also be freely placed and superimposed as character strings in any of the four corners of the radiograph display area 504.

The optical image window display toggle button 506 is a selection object that can be pressed to manually toggle the displayed or hidden (on/off) state of the optical image window 550.

The end examination button 507 is a selection object that can be pressed to end the current examination.

In the optical image window 550, an optical image acquired by the optical image acquisition unit 203 and the result of the analysis by the optical image analysis unit 205 are displayed along with buttons and the like for controlling the optical image.

In the optical image window 550, an analysis is performed for the currently selected “Protocol-A” radiography details. The optical image window 550 is superimposed onto the radiograph display area 504.

The optical image window 550 includes an optical image display area 551, camera information 552, an optical image analysis in progress icon 553, an optical image display toggle button 554, a rotate optical image button 555, and an optical image analysis result 556.

In the optical image display area 551, an optical image acquired by the optical image acquisition unit 203 is displayed. Although image processing is not performed on the optical image in FIG. 5, image processing may be performed to make the optical image easier for the user to see.

In the camera information 552, information about the optical image capture device 40 is displayed.

The optical image analysis in progress icon 553 is information indicating whether or not image analysis is being executed by the optical image analysis unit 205. For example, by displaying the optical image analysis in progress icon 553 while the optical image analysis process is being executed, the icon 553 is displayed to visually notify the user that optical image analysis is in progress. When the optical image analysis process is not in progress, the icon 553 may be hidden or grayed out.

The optical image display toggle button 554 is a selection object that can be used to manually toggle the displayed or hidden state of the optical image in the optical image display area 551.

The rotate optical image button 555 is a selection object that can be used to rotate the optical image displayed in the optical image display area 551. In the present embodiment, only left rotation is available, but right rotation may also be added, and a form for selecting the rotation angle may also be implemented.

The optical image analysis result 556 is a message area (notification area) for displaying an image analysis result (status information about the subject) provided by the optical image analysis unit 205.

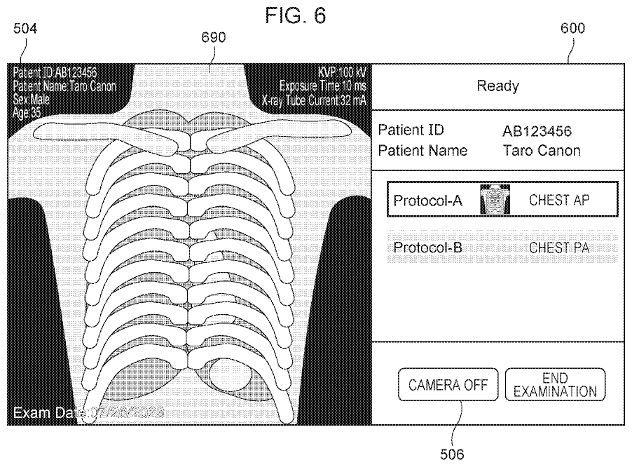

When proceeding to the next step from the stage illustrated in FIG. 5, the display transitions to the screen in FIG. 6. FIG. 6 is a diagram illustrating an example of a display screen immediately after radiography in an imaging management application.

When radiography is executed, a screen 600 is displayed. On the screen 600, a radiograph 690 is displayed in the radiograph display area 504. Accordingly, the optical image window 550 that had been superimposed onto the radiograph display area 504 is hidden. In addition, the display information of the optical image window display toggle button 506 changes from “Camera ON” to “Camera OFF”.

Note that still imaging is given as an example herein, but in the case where the radiography details include fluoroscopy (dynamic imaging), the screen in FIG. 6 may also be displayed while radiography is underway. This allows for constant checking of fluoroscopic images.

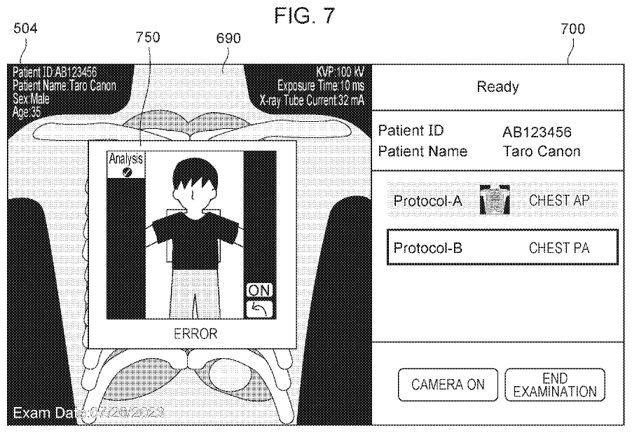

When proceeding to the next step from the stage illustrated in FIG. 6, the display transitions to the screen in FIG. 7. FIG. 7 is a diagram illustrating an example of a display screen during imaging standby in an imaging management application.

When the next set of radiography details are selected in the examination information 503, the system prepares to start examination again and the screen 700 is displayed. On the screen 700, “Protocol-B” is in the selected state. On the screen 700, an optical image window 750 is superimposed on top of a radiograph 690 in the radiograph display area 504. In the optical image window 750, an analysis is performed for “Protocol-B”.

### Screen Display Control

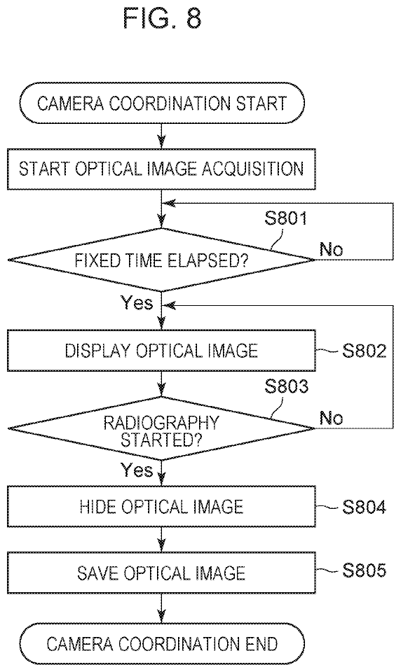

FIG. 8 will be used to describe a flow from when an examination is started to when the examination is ended. FIG. 8 is a diagram illustrating a flowchart of optical image display. Control corresponding to this flowchart is executed in the control device 20. Specifically, the control is realized by having the CPU 301 provided in the control device 20 load the computer program 3031 stored in the ROM 303 or the like into the RAM 302 and act as corresponding functions.

In step S801, the control device 20 measures the elapsed time since radiography details were selected in the imaging management application. Once a fixed time elapses, the flow proceeds to S802.

The fixed time indicated herein may be changed between the first instance of radiography and the second and subsequent instances of radiography in the current examination. Time more appropriate to the workflow may be set, such as setting time to check patient information in the first instance of radiography and setting time to check the radiographs in the second and subsequent instances.

In S802, the optical image acquisition unit 203 acquires an optical image captured by the optical image capture device 40. The optical image display control unit 204 then provides image information to the display control unit 209, thereby causing the optical image to be displayed on the display unit 25. This causes a screen like the screen 500 to be displayed.

In S803, the control device 20 checks whether radiography has started. The flow waits (NO) until radiography is started, and once radiography is started (YES), the flow proceeds to S804. The start of radiography means that the radiation detecting device 10 has started a radiation accumulating operation, for example. The start may be defined as the radiation generating device 30 having started emitting radiation toward the radiation detecting device 10, as the control device 20 having started receiving an image from the radiation detecting device 10, as the control device 20 having completed preparations whereby an image can be displayed on the display unit 25, or the like.

In S804, the display control unit 209 automatically hides the optical image displayed on the display unit 25 and displays a screen like the screen 600.

In S805, the image storage unit 208 saves the optical image. When saving the optical image, the optical image is saved in association with the radiograph acquired by the radiograph acquisition unit 201 and the examination information managed by the examination information management unit 207.

Through the above steps, radiography of the imaging target ends, and the flow proceeds to the next instance of radiography. If the current examination includes multiple instances of radiography, the next set of radiography details are selected and a screen similar to the screen 700 for the next set of radiography details is displayed. This process is repeated a number of times equal to number of registered sets of radiography details.

### Effects

As described in the first embodiment above, the display can be suited to the workflow by displaying the optical image when the user wants to check the optical image, and by hiding the optical image the user wants to check the radiograph or other display. By displaying the optical image at a smaller size than the radiograph, the superimposing of the optical image does not obscure information that the user would want to check, such as additional information in the four corners of the radiograph, and therefore the display can be further suited to the workflow. Even if content that the user would not check in an ordinary workflow is obscured, the optical image can be toggled to be displayed or hidden, thereby also accommodating workflows other than an ordinary workflow. Furthermore, by saving the optical image at the start of radiography, and by saving the optical image in association with the radiograph and the examination information, it is possible to leave a record of the situation at the time of the radiography.

### Second Embodiment

The first embodiment illustrates an example in which the optical image window 550 is superimposed onto a portion of the radiograph display area 504. In contrast, the second embodiment is characterized by utilizing the entirety of the radiograph display area 504 as the optical image window 550. Note that the configuration for realizing the second embodiment is similar to that of the first embodiment except for the portion related to the abovementioned characteristic portion. Accordingly, the similar portions of the configuration are denoted and described using similar signs, and a detailed description of such portions is omitted.

### Display of Optical Image

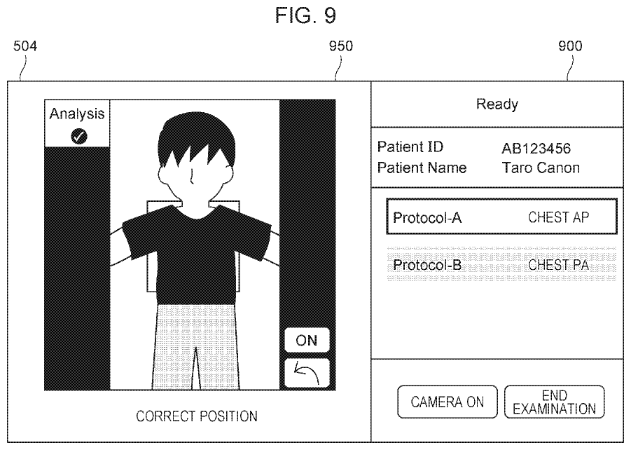

FIG. 9 will be used to describe the display of the optical image. FIG. 9 is a diagram illustrating an optical image display example in an imaging management application.

In the present embodiment, the status of the examinee during radiography is captured in an image by the optical image capture device 40, and the optical image obtained thereby is used for status monitoring.

At the start of a radiographic examination, a screen 900 is displayed on the GUI of the imaging management application. Compared to the screen 500, the screen 900 differs in that an optical image window 950 corresponding to the optical image window 550 extends over the entirety of the radiograph display area 504. Enlarging the window makes the optical image easier to see, which facilitates checking of the status of radiography.

During radiography and immediately afterward, a screen 1000 is displayed on the GUI of the imaging management application. Compared to the screen 600, the screen 1000 differs in that an optical image window 1050 is displayed out of (outside) the radiograph display area 504. Displaying the optical image in this way makes it possible to monitor the status of the examinee via the optical image while also checking the radiograph during radiography and immediately afterward.

### Screen Display Control

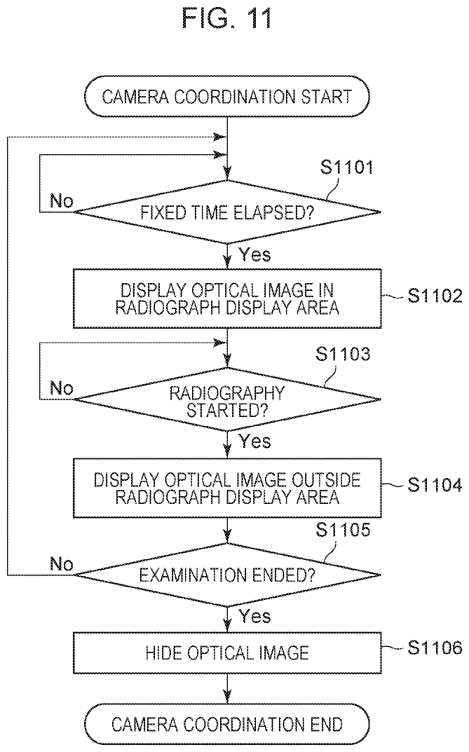

FIG. 11 will be used to describe a flow from when an examination is started to when the examination is ended. FIG. 11 is a diagram illustrating a flowchart of optical image display. Control corresponding to this flowchart is executed in the control device 20. Specifically, the control is realized by having the CPU 301 provided in the control device 20 load the computer program 3031 stored in the ROM 303 or the like into the RAM 302 and act as corresponding functions.

In S1101, the control device 20 measures the elapsed time since the previous process. Once a fixed time elapses, the flow proceeds to S1102.

In S1102, the control device 20 displays an optical image in the radiograph display area 504.

In S1103, the control device 20 checks whether radiography has started. If radiography has started, the flow proceeds to S1104.

In S1104, the control device 20 displays an optical image outside the radiograph display area 504.

In S1105, the control device 20 checks whether the current examination has ended. If the examination has not ended, the flow proceeds to S1101. If the examination has ended, the flow proceeds to S1106.

In S1106, the control device 20 hides the optical image and ends the examination.

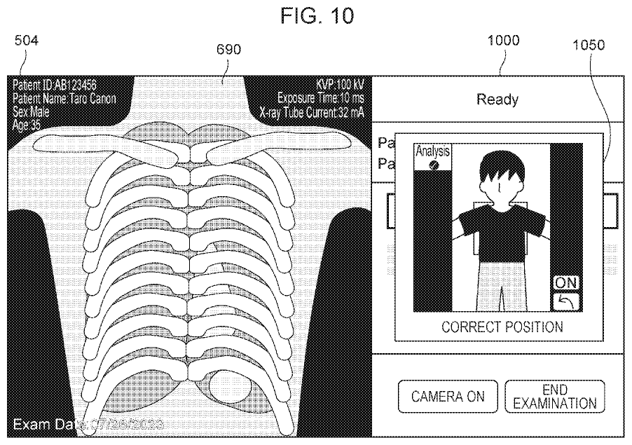

According to this flow, the optical image is set to the displayed state from when the examination is started to when the examination is ended. Note that the fixed time in S1101 may be changed between the first instance of radiography and the second and subsequent instances of radiography in the current examination, similarly to the first embodiment. The fixed time in S1101 may also be set to 0 seconds. Like the display example illustrated in FIG. 10, since the optical image window 1050 is displayed out of (outside) the radiograph display area 504, the radiograph can be viewed. Also, since the examinee information can be checked via the character strings in the four corners of the radiograph display area 504, the examinee information 502 may be obscured even when checking the examinee after the current examination starts. Moreover, since the optical image does not interfere with the operator's ability to check the radiograph after radiography, the display state of the optical image can be continued even after radiography.

### Effects

As described in the second embodiment above, the output can be further suited to the workflow by switching the display position, size, and the like of the optical image according to the step of the process.

### Third Embodiment

In the first and second embodiments, the image captured by the optical image capture device 40 is itself displayed as the optical image. The third embodiment is characterized by performing image processing on the optical image to be displayed. Note that the configuration for realizing the third embodiment is similar to that of the first embodiment except for the portion related to the abovementioned characteristic portion. Accordingly, the similar portions of the configuration are denoted and described using similar signs, and a detailed description of such portions is omitted.

### Display of Optical Image

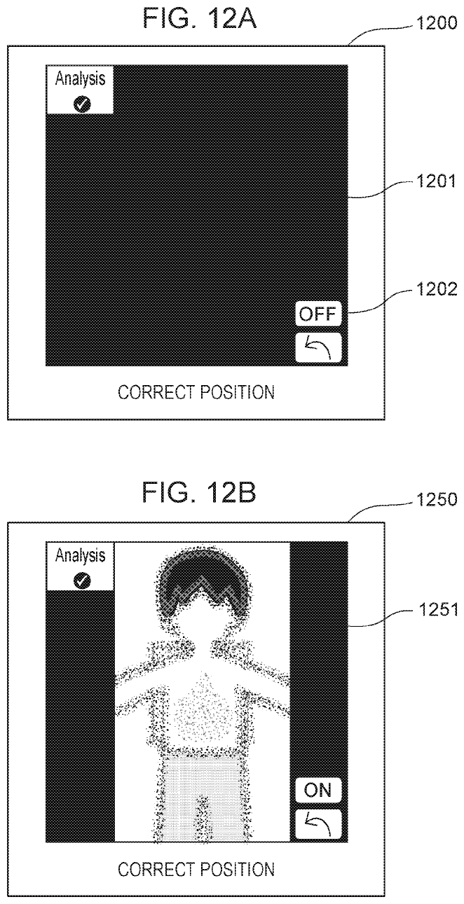

FIG. 12B will be used to describe the display of the optical image. FIG. 12B is a diagram illustrating a situation in which image processing is applied to an optical image.

In an optical image window 1250, an image 1251 obtained by performing a mosaicing process (image alteration) is displayed as the optical image. This image processing is performed by the image processing unit 202.

In the image 1251, a mosaicing process is performed as the alteration of the optical image, but the optical image may also be altered using figures such as curves and rectangles to make the examinee unidentifiable. The alteration may also be limited to privacy-related portions, and the alteration may also be performed via image cropping, masking, or other processing. Such portions may also be replaced with images not based on the optical image, such as solid-color surfaces. Alternatively, an optical image 1201 in an optical image window 1200 may be set to hidden, as in FIG. 12A. FIG. 12A is a diagram illustrating a situation in which the display of an optical image is turned off. When the optical image 1201 is set to hidden, the display of an optical image display toggle button 1202 changes from “ON” to “OFF”. Note that even if the optical image 1201 is hidden, the analysis of the optical image is performed and the user is notified of the result, thereby enabling the user to grasp the situation.

### Screen Display Control

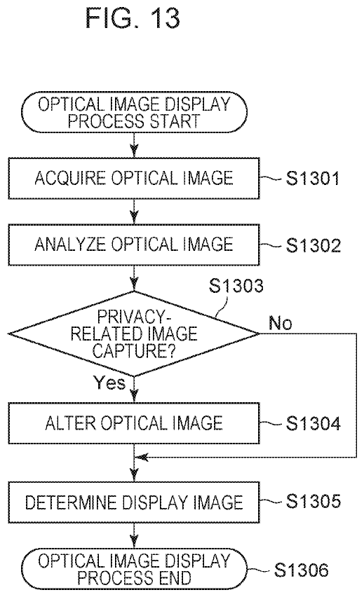

FIG. 13 will be used to describe a detailed flow of the optical image display process (S802, S1102).

FIG. 13 is a diagram illustrating a flowchart of optical image display. Control corresponding to this flowchart is executed in the control device 20. Specifically, the control is realized by having the CPU 301 provided in the control device 20 load the computer program 3031 stored in the ROM 303 or the like into the RAM 302 and act as corresponding functions.

In S1301, the control device 20 acquires an optical image for determining privacy and then advances the process to S1302.

In S1302, the control device 20 analyzes the optical image and then advances the process to S1303.

In S1303, the control device 20 determines whether the current image capture is privacy-related. For example, the determination may be based on whether the image capture is performed with the examinee partially or fully undressed, whether the area of the body to be imaged is the hip joint or the like, or whether the image capture could result in private parts (genitalia, pubic region) being visible in the optical image. If the image capture is privacy-related (YES), the flow proceeds to S1304, otherwise the flow proceeds to S1305 (NO).

In S1304, the control device 20 alters the optical image due to being privacy-related.

In S1305, the control device 20 determines the optical image to be displayed. If the flow has passed through S1304, the altered optical image is determined. The image determined at this point becomes the image displayed in the optical image display process (S802, S1102).

Note that in the present embodiment, the analysis result regarding the optical image is used to determine privacy, but other information may also be used to determine privacy. For example, the examination information (radiography details) inputted in advance by the user may also be used to determine whether or not privacy-related optical image capture is performed.

### Effects

As described in the third embodiment above, the output can be further suited to the workflow by performing privacy-related handling.

### Fourth Embodiment

The first embodiment describes an example in which the analysis result regarding the optical image is provided as a brief notification. The fourth embodiment describes an example in which the analysis result regarding the optical image is provided as a detailed notification. Note that the configuration for realizing the fourth embodiment is similar to that of the first embodiment except for the portion related to the abovementioned characteristic portion. Accordingly, the similar portions of the configuration are denoted and described using similar signs, and a detailed description of such portions is omitted.

### Notification of Analysis Result

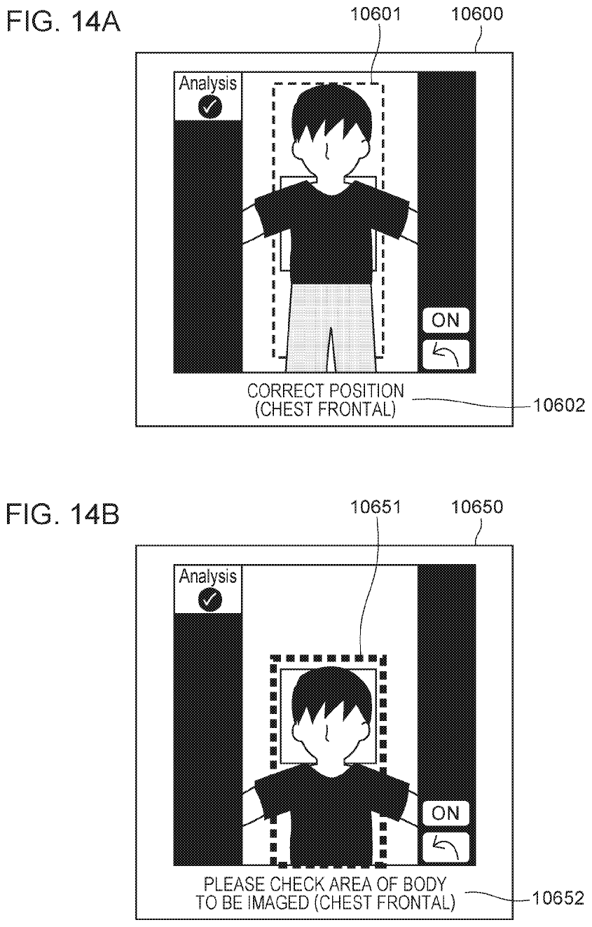

Next, the notification of the analysis result will be described in detail using FIGS. 14A and 14B.

FIG. 14A is a diagram illustrating an analysis result notification example 1 in an imaging management application.

FIG. 14B is a diagram illustrating an analysis result notification example 2 in an imaging management application.

When an optical image is analyzed, first, an optical image analysis target detection frame 10601 is displayed in a screen state 10600 of the optical image window 550 described earlier. The optical image analysis target detection frame 10601 is a frame indicating that a single human body was successfully detected and also indicating the detection area thereof (target area of determination) in the optical image analysis unit 205. For example, in the optical image analysis unit 205, the optical image analysis target detection frame 10601 is hidden when a human body is not detected, and the optical image analysis target detection frame 10601 is displayed when a human body is detected. With this arrangement, the user can receive a visual notification of the human detection result and the area thereof in the optical image analysis. If a human body is detected in an incorrect position, an emphasized optical image analysis target detection frame 10651 may be displayed to indicate as much in a screen state 10650 of the optical image window 550 described earlier. That is, the manner of displaying the optical image analysis target detection frame may be changed depending on the status of human body detection.

Also, in the screen state 10600, a notification 10602 is provided as the optical image analysis result 556. The notification 10602 is a notification in the case where a human body is detected correctly in correspondence with the examination information. A message stating “This is the correct position” and the information “Chest frontal” as the current radiography details (imaging pose) are displayed as the notification 10602. Through this notification, the user can understand that the human body is in the correct position with respect to the scheduled radiography details.

On the other hand, in the screen state 10650, a notification 10652 is provided as the optical image analysis result 556. The notification 10652 is a notification in the case where a human body is detected in an incorrect position in correspondence with the examination information. A message stating “Please check area of body to be imaged” and the information “Chest frontal” as the current radiography details are displayed as the notification 10652. Through this notification, the user can understand that the human body is not in the correct position (inconsistent, error) with respect to the scheduled radiography details.

### Examination Process

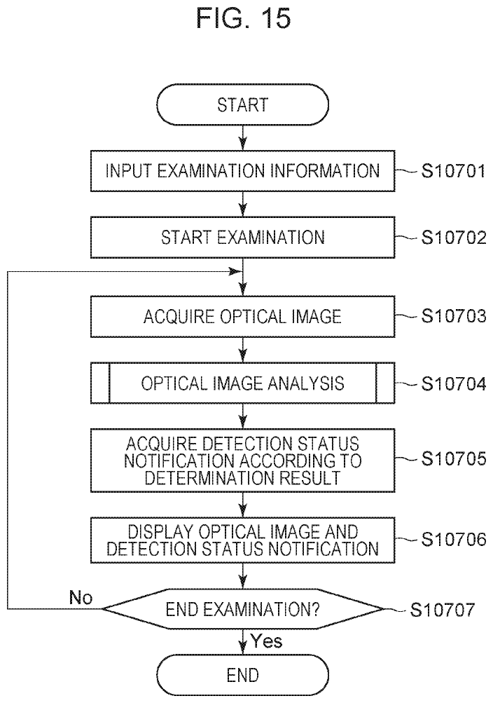

Next, FIG. 15 will be used to describe an examination process. FIG. 15 is a diagram illustrating a flowchart of an examination process. Control corresponding to this flowchart is executed in the control device 20.

Specifically, the control is realized by having the CPU 301 provided in the control device 20 load the computer program 3031 stored in the ROM 303 or the like into the RAM 302 and act as corresponding functions.

In S10701, the examination information management unit 207 prompts the user to select one from among the plurality of examination information acquired from the operation unit 26, and sets the selected examination information as the examination target. This process is realized by, for example, displaying the acquired plurality of examination information in the form of a list, accepting operating input by the user to select one piece of examination information from the list, and setting the selected examination information as the examination target. Note that the user may also enter examination information directly from the operation unit 26.

In S10702, the control device 20 starts an examination by transmitting a signal causing the radiation detecting device 10 to transition to a preparatory state in accordance with the set examination information.

In response to this signal, for example, the radiation detecting device 10 uses a main control circuit to control a bias power supply and apply a bias voltage to a two-dimensional image sensor. After that, initialization is performed, in which an image signal is read out from a pixel array using a drive circuit to read out a dark current signal accumulated in the pixels. After initialization finishes, the radiation detecting device 10 transmits, to the control device 20, status information indicating that preparations for obtaining a radiograph are complete. In addition, the control device 20 (examination information management unit 207) sets operating parameters (such as the tube voltage) of the radiation detecting device 10 on the basis of the examination information that was selected in S10701. The control device 20, upon being notified that radiography preparations are complete via the status information from the radiation detecting device 10, gives exposure permission in a notification to the radiation detecting device 10.

In S10703, the optical image acquisition unit 203 acquires an optical image captured by the optical image capture device 40.

In S10704, the optical image analysis unit 205 performs optical image analysis using the optical image acquired from the optical image acquisition unit 203 and the examination information acquired from the examination information management unit 207. To analyze the optical image, an inference process using machine learning may be performed by the inference processing unit 206, for example. The images to be inputted into the optical image analysis unit 205 may be all images that the optical image acquisition unit 203 has acquired, or the images may be decimated according to the analysis details before being inputted into the optical image analysis unit 205. The details of S10704 will be described later.

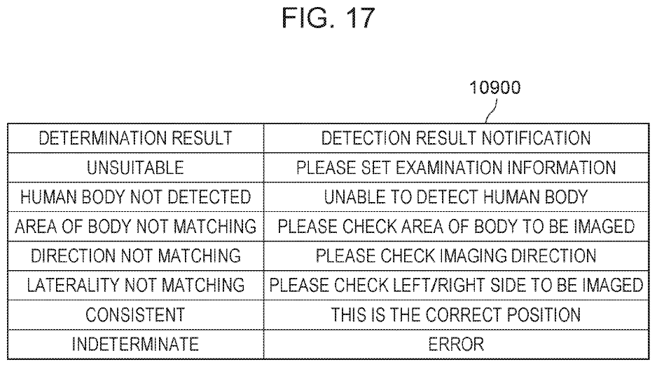

In S10705, the control device 20 acquires the result of the determination performed in S10704 and acquires the information of a corresponding detection status notification. An example of the correspondence between the result of the determination and the detection status notification is illustrated by the table 10900 in FIG. 17. FIG. 17 is a diagram illustrating the correspondence between determination results from image analysis and detection result notifications.

In S10706, the optical image display control unit 204 uses the optical image acquired by the optical image acquisition unit 203 and the analysis result from the optical image analysis unit 205 to control and provide display content to the display control unit 209. This causes the controlled display content to be displayed on the display unit 25. In the optical image display control unit 204, the optical image and the analysis result may each be displayed in any way, and the display may be controlled according to settings of the control device 20 and settings of the examination information management unit 207. For example, in the case of not performing optical image analysis, only the optical image may be displayed on the display unit 25.

### Optical Image Analysis Process

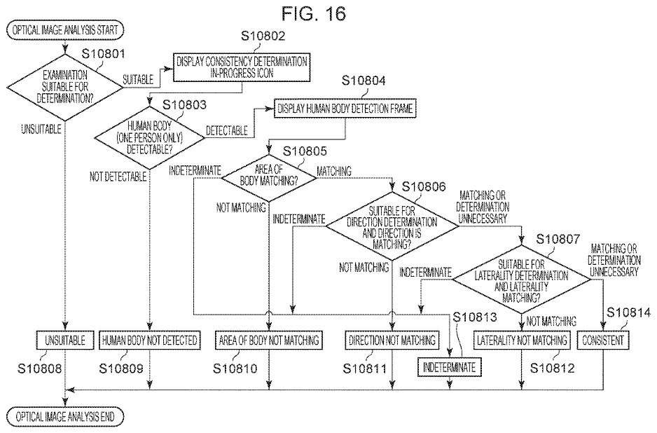

Next, the optical image analysis process (process in S10704) by the optical image analysis unit 205 will be described with reference to FIG. 16. FIG. 16 is a diagram illustrating a flowchart of an optical image analysis process.

Control corresponding to this flowchart is executed in the control device 20. Specifically, the control is realized by having the CPU 301 provided in the control device 20 load the computer program 3031 stored in the ROM 303 or the like into the RAM 302 and act as corresponding functions. The order of the steps may be rearranged freely. Also, the processing in some steps may be omitted. Also, processing to store display information may be omitted.

In S10801, the optical image analysis unit 205 determines whether or not the examination information acquired by the examination information management unit 207 is examination information suitable for optical image analysis. For example, if the examination information does not contain sufficient information to analyze whether the optical image conforms to the details of the specified examination in the optical image analysis, the examination information is determined to be unsuitable. Specifically, the examination information is determined to be unsuitable in cases such as when analysis for determining the area of the body is to be performed but the examination information does not specify the area of the body, or specifies an area of the body that the optical image analysis unit 205 is incapable of analyzing. If the examination information is determined to be unsuitable, the flow proceeds to the “Unsuitable” determination result in S10808.

In S10802, display information is stored in the determination result to display a consistency determination in-progress icon indicating that the examination is suitable for optical image analysis.

In S10803, the optical image analysis unit 205 determines whether the subject to be analyzed is included in the optical image acquired from the optical image acquisition unit 203. The method of detecting the subject may use image recognition techniques involving machine learning. At this time, if the optical image contains not only the body of the patient to be imaged but also the body of someone other than the patient, such as the radiologist making the preparations for radiography, it may be considered that the appropriate subject cannot be detected, and a result of “Not detectable” may be determined. If a result of not detectable is determined, the flow transitions to the “Human body not detected” determination result in S10809.

In S10804, display information is stored in the determination result to display a human body detection frame indicating that the examination is suitable for optical image analysis.

In S10805, the optical image analysis unit 205 determines whether the area of the body in the examination information acquired by the examination information management unit 207 and the area of the body in the analysis result regarding the optical image acquired from the optical image acquisition unit 203 are matching. The method of analyzing the area of the body may use image recognition techniques involving machine learning. If the analysis result is determined to be not matching, the flow transitions to the “Area of body not matching” determination result in S10810. In the indeterminate case in which the analysis result cannot be determined to be matching or not matching, the flow transitions to the “Indeterminate” determination result in S10813.

In S10806, the optical image analysis unit 205 determines whether the direction in the examination information acquired by the examination information management unit 207 and the direction in the analysis result regarding the optical image acquired from the optical image acquisition unit 203 are matching. Depending on the examination information, this determination may be unnecessary. The method of analyzing the direction may use image recognition techniques involving machine learning. If the analysis result is determined to be not matching, the flow transitions to the “Direction not matching” determination result in S10811. In the indeterminate case in which the analysis result cannot be determined to be matching or not matching, the flow transitions to the “Indeterminate” determination result in S10813.

In S10807, the optical image analysis unit 205 determines whether the laterality of the body in the examination information acquired by the examination information management unit 207 and the laterality in the analysis result regarding the optical image acquired from the optical image acquisition unit 203 are matching. Depending on the examination information, this determination may be unnecessary. The method of analyzing the laterality may use image recognition techniques involving machine learning. If the analysis result is determined to be not matching, the flow transitions to the “Laterality not matching” determination result in S10812. In the indeterminate case in which the analysis result cannot be determined to be matching or not matching, the flow transitions to the “Indeterminate” determination result in S10813.

If the analysis result is matching, the flow transitions to the “Consistent” determination result in S10814.

S10808 to S10814 illustrate states of the determination result from the optical image analysis unit 205.

The optical image display control unit 204 can control the display content in the optical image window 550 according to each determination result. Specifically, the optical image display control unit 204 can control the notification content as illustrated by the table 10900 in FIG. 17.

Note that the notification provided by the optical image analysis result 556 is not limited to a textual message. Besides text, figures, symbols, colors, or the like may also be used to provide notifications such that analysis results can be distinguished from each other.

### Effects

As described in the embodiment above, by determining the consistency between the specified radiography procedure and the actual positioning of the examinee, the user can be made aware of possible rejection in advance. Accordingly, it is possible to reduce rejection and improve workflow efficiency.

### Fifth Embodiment

The first embodiment describes an example that does not distinguish between a plurality of optical image capture devices, a plurality of radiation generating devices, and a plurality of radiation detecting devices. The fifth embodiment describes an example that distinguishes between a plurality of radiation generating devices and a plurality of radiation detecting devices. Note that the configuration for realizing the fifth embodiment is similar to that of the first embodiment except for the portion related to the abovementioned characteristic portion. Accordingly, the similar portions of the configuration are denoted and described using similar signs, and a detailed description of such portions is omitted.

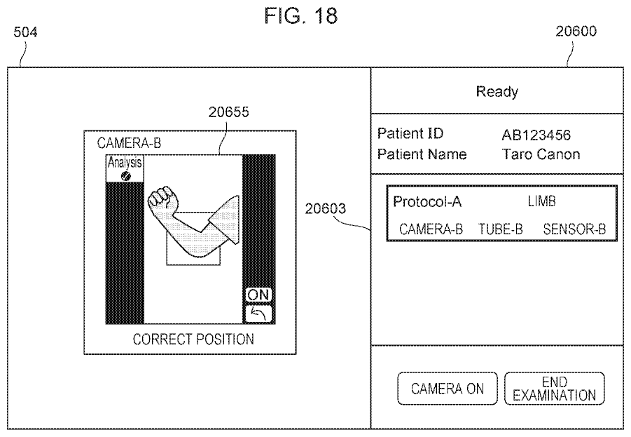

FIG. 18 will be used to describe the display of the optical image. FIG. 18 is a diagram illustrating an optical image display example 2 in an imaging management application.

On a screen 20600, “Protocol-A” radiography information is registered in examination information 20603. A comparison between the examination information 20603 and the examination information 503 reveals that this radiography information differs in that the imaging target is “Limb” rather than “Chest”. In association with the above, “Camera-A”, “Tube-A”, and “Sensor-A” written in the examination information 503 are changed to “Camera-B”, “Tube-B”, and “Sensor-B” in the examination information 20603. “Camera-A”, “Tube-A”, and “Sensor-A” correspond to the optical image capture device 40A, the radiation generating device 30A, and the radiation detecting device 10A, respectively. “Camera-B”, “Tube-B”, and “Sensor-B” correspond to the optical image capture device 40B, the radiation generating device 30B, and the radiation detecting device 10B, respectively.

These devices to be used in radiography are changed automatically in coordination with the radiography details selected in the examination information 503 or the examination information 20603. Furthermore, in the present embodiment, these coordinated devices are displayed as a part of the examination information to allow for confirmation by the user. Also, an optical image 20655 displayed in the optical image display area 551 was captured by Camera-B, and thus camera information 20652 also switches to “Camera-B”.

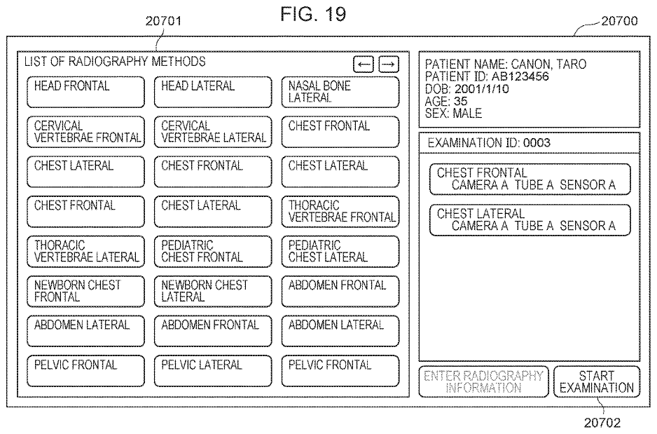

The radiography information registered in the examination information 503 or the examination information 20603 is specified in advance on a screen 20700 illustrated in FIG. 19, prior to radiography. FIG. 19 is a diagram illustrating a radiography method settings screen in an imaging management application.

The screen 20700 includes a radiography method list area 20701 and a start examination button 20702. By selecting a radiography method from the list and then selecting the start examination button 20702, the user can cause a screen with registered examination information to be displayed like the screen 500 or the screen 20600. When a radiography method is selected from the radiography method list area 20701, the devices to use are determined on the basis of the combination of device configuration information about the radiographic imaging system 1 and the imaging method. Information about the determined devices to use is displayed as radiography information. In addition, the image orientation (angle information) of the optical image capture device 40 is also registered in the radiography information. Geometric transformations and the like of the image are applied automatically on the basis of the registered image orientation information, and thus the optical image is displayed in the appropriate orientation in the optical image display area 551 in the default state.

### Determination of Devices to Use and Optical Image Geometry

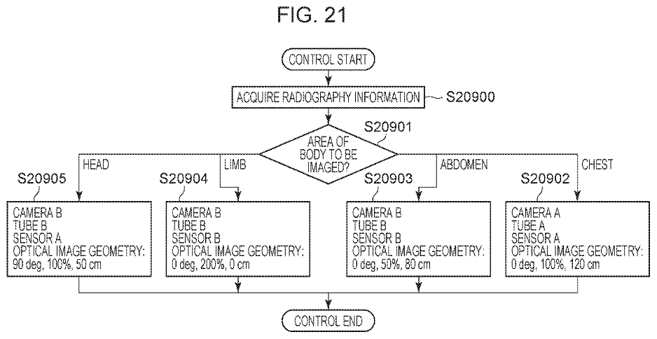

Next, FIG. 21 will be used to describe control related to the determination of the devices to use and the image orientation.

FIG. 21 is a diagram illustrating a flowchart of radiography information management method (a).

In S20900, the control device 20 accepts the input of radiography information from the user and acquires the radiography information.

In S20901, the control device 20 determines the inputted area of the body to be imaged. For simplicity, the following describes the determination being made for the following four areas of the body: “Head”, “Limb”, “Abdomen”, and “Chest”. However, the area of the body to be imaged may also be classified more finely. If the area of the body to be imaged is “Chest”, the process proceeds to S20902. If the area of the body to be imaged is “Abdomen”, the process proceeds to S20903.

If the area of the body to be imaged is “Limb”, the process proceeds to S20904. If the area of the body to be imaged is “Head”, the process proceeds to S20905.

In S20902, the control device 20 determines “Camera A”, “Tube A”, and “Sensor A” as the devices to use and determines “Rotation 0 deg”, “Scale 100%”, and “Height 120 cm” as the optical image geometry (geometry information), on the basis of the table of radiography information management method (a). “Rotation 0 deg” is rotation information, “Scale 100%” is enlargement or reduction information, and “Height 120 cm” is center coordinate information.

In S20903, the control device 20 determines “Camera B”, “Tube B”, and “Sensor B” as the devices to use and determines “Rotation 0 deg”, “Scale 50%”, and “Height 80 cm” as the optical image geometry, on the basis of the table of radiography information management method (a).

In S20904, the control device 20 determines “Camera B”, “Tube B”, and “Sensor B” as the devices to use and determines “Rotation 0 deg”, “Scale 200%”, and “Height 0 cm” as the optical image geometry, on the basis of the table of radiography information management method (a).

In S20905, the control device 20 determines “Camera B”, “Tube B”, and “Sensor A” as the devices to use and determines “Rotation 90 deg”, “Scale 100%”, and “Height 50 cm” as the optical image geometry, on the basis of the table of radiography information management method (a).

In the above, the area of the body to be imaged is given as an example, but the devices to use and the optical image geometric transformation may also be determined by using patient information such as sex, body type, weight, height, and age.

### Effects

According to the fifth embodiment above, the devices to use and the optical image geometry can be determined by selecting the area of the body to be imaged. Accordingly, an optical image from an appropriate optical image capture device can be displayed at an appropriate angle and magnification in the optical image display area 551 of the optical image window 550.

Additionally, position information such as coordinate information relative to the ground can also be designated as information on the optical image geometry. Therefore, in the case of an optical image capture device capable of panning or an optical image capture device that captures wide-angle images, it is also possible to capture the area of the body to be imaged in the center of the optical image.

### Sixth Embodiment

In the fifth embodiment, the devices to use and the optical image geometry are determined by selecting the area of the body to be imaged. In contrast, in the sixth embodiment, the devices to use and the optical image geometry are determined by selecting an imaging type. Note that the configuration for realizing the sixth embodiment is similar to that of the fifth embodiment except for the portion related to the abovementioned characteristic portion. Accordingly, the similar portions of the configuration are denoted and described using similar signs, and a detailed description of such portions is omitted.

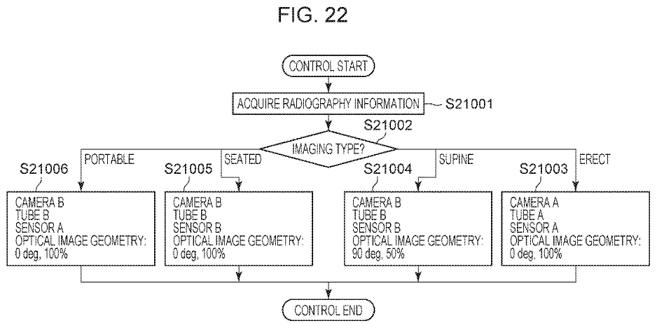

FIG. 22 is a diagram illustrating a flowchart of radiography information management method (b).

In S21001, the control device 20 accepts the input of radiography information from the user and acquires the radiography information. Unlike the fifth embodiment, in which the area of the body to be imaged is acquired, in the sixth embodiment, it is assumed that the imaging type is acquired. That is, it is assumed that the imaging type is selected in advance on an imaging type selection screen (not illustrated).

In S21002, the control device 20 determines the inputted imaging type. For simplicity, the following describes the determination being made for the following four imaging types: “Portable”, “Seated”, “Supine”, and “Erect”. However, the imaging type may also be classified more finely. If the imaging type is “Erect”, the process proceeds to S21003. If the imaging type is “Supine”, the process proceeds to S21004. If the imaging type is “Seated”, the process proceeds to S21005. If the imaging type is “Portable”, the process proceeds to S21006.

In S21003, the control device 20 determines “Camera A”, “Tube A”, and “Sensor A” as the devices to use and determines “Rotation 0 deg” and “Scale 100%” as the optical image geometry, on the basis of the table of radiography information management method (b).

In S21004, the control device 20 determines “Camera B”, “Tube B”, and “Sensor B” as the devices to use and determines “Rotation 90 deg” and “Scale 50%” as the optical image geometry, on the basis of the table of radiography information management method (b).

In S21005, the control device 20 determines “Camera B”, “Tube B”, and “Sensor B” as the devices to use and determines “Rotation 0 deg” and “Scale 100%” as the optical image geometry, on the basis of the table of radiography information management method (b).

In S21006, the control device 20 determines “Camera B”, “Tube B”, and “Sensor A” as the devices to use and determines “Rotation 0 deg” and “Scale 100%” as the optical image geometry, on the basis of the table of radiography information management method (b).

### Effects

According to the sixth embodiment above, the devices to use and the optical image geometry can be determined by selecting the imaging type. Accordingly, an optical image from an appropriate optical image capture device can be displayed at an appropriate angle and magnification in the optical image display area 551 of the optical image window 550.

### Seventh Embodiment

In the sixth embodiment, the devices to use and the optical image geometry are determined by selecting the imaging type. The seventh embodiment describes a radiography information management method for an environment in which “Tube A” and “Camera A” are always operated together as a set and “Tube B” and “Camera B” are always operated together as a set. This embodiment is used in cases where close coordination with the radiation tube is desired, such as when the optical image capture device is installed near the radiation tube. Note that the configuration for realizing the seventh embodiment is similar to that of the sixth embodiment except for the portion related to the abovementioned characteristic portion. Accordingly, the similar portions of the configuration are denoted and described using similar signs, and a detailed description of such portions is omitted.

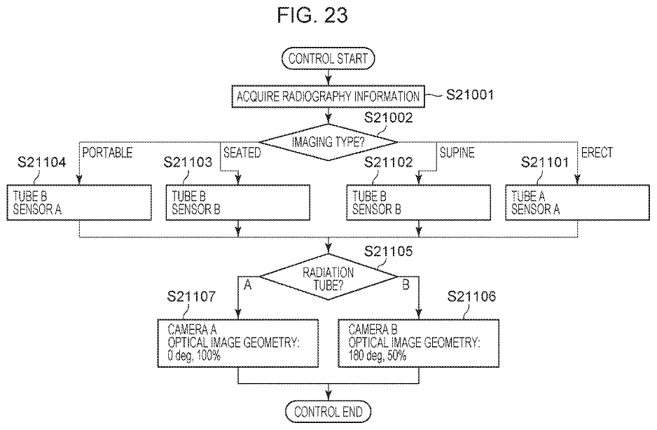

FIG. 23 is a diagram illustrating a flowchart of radiography information management method (c).

In S21001, the control device 20 acquires radiography information in a manner similar to the sixth embodiment.

In S21002, the control device 20 acquires the imaging type in a manner similar to the sixth embodiment. If the imaging type is “Erect”, the process proceeds to S21101. If the imaging type is “Supine”, the process proceeds to S21102. If the imaging type is “Seated”, the process proceeds to S21103. If the imaging type is “Portable”, the process proceeds to S21104.

In S21101, the control device 20 determines “Camera A”, “Tube A”, and “Sensor A” as the devices to use, on the basis of the table of radiography information management method (c).

In S21102, the control device 20 determines “Camera B”, “Tube B”, and “Sensor B” as the devices to use, on the basis of the table of radiography information management method (c).

In S21103, the control device 20 determines “Camera B”, “Tube B”, and “Sensor B” as the devices to use, on the basis of the table of radiography information management method (c).

In S21104, the control device 20 determines “Camera B”, “Tube B”, and “Sensor A” as the devices to use, on the basis of the table of radiography information management method (c).

In S21105, the control device 20 determines the radiation tube to use. If the radiation tube to use is “Tube B”, the process proceeds to S21106. If the radiation tube to use is “Tube A”, the process proceeds to S21107.

In S21106, the control device 20 determines “Camera B” as the device to use and determines “Rotation 180 deg” and “Scale 50%” as the optical image geometry, on the basis of the table of radiography information management method (c).

In S21107, the control device 20 determines “Camera B” as the device to use and determines “Rotation 0 deg” and “Scale 100%” as the optical image geometry, on the basis of the table of radiography information management method (c).

### Effects

According to the seventh embodiment above, the devices to use and the optical image geometry can be determined by selecting the imaging type. Accordingly, an optical image from an appropriate optical image capture device can be displayed at an appropriate angle and magnification in the optical image display area 551 of the optical image window 550.

### Eighth Embodiment

In the sixth embodiment, the devices to use and the optical image geometry are determined by selecting the imaging type. The eighth embodiment describes a radiography information management method for an environment in which “Sensor A” and “Camera A” are always operated together as a set and “Sensor B” and “Camera B” are always operated together as a set. This embodiment is used in cases where close coordination of the optical image capture device with the radiation detecting device is desired, such as when it is desired to display the radiograph and the optical image in the same way or when it is desired to align the optical image with the scanning or reading direction of the radiation detecting device. Note that the configuration for realizing the eighth embodiment is similar to that of the sixth embodiment except for the portion related to the abovementioned characteristic portion. Accordingly, the similar portions of the configuration are denoted and described using similar signs, and a detailed description of such portions is omitted.

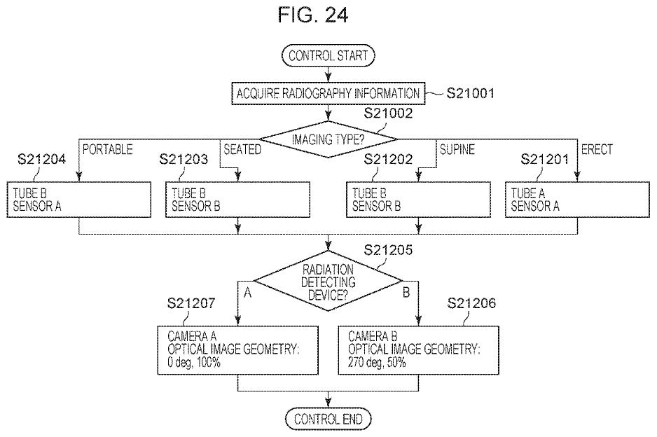

FIG. 24 is a diagram illustrating a flowchart of radiography information management method (d).

In S21001, the control device 20 acquires radiography information in a manner similar to the sixth embodiment.

In S21002, the control device 20 acquires the imaging type in a manner similar to the sixth embodiment. If the imaging type is “Erect”, the process proceeds to S21201. If the imaging type is “Supine”, the process proceeds to S21202. If the imaging type is “Seated”, the process proceeds to S21203. If the imaging type is “Portable”, the process proceeds to S21204.

In S21201, the control device 20 determines “Tube A” and “Sensor A” as the devices to use, on the basis of the table of radiography information management method (d).

In S21202, the control device 20 determines “Tube B” and “Sensor B” as the devices to use, on the basis of the table of radiography information management method (d).

In S21203, the control device 20 determines “Tube B” and “Sensor B” as the devices to use, on the basis of the table of radiography information management method (d).

In S21204, the control device 20 determines “Tube B” and “Sensor A” as the devices to use, on the basis of the table of radiography information management method (d).

In S21205, the control device 20 determines the radiation detecting device to use. If the radiation detecting device to use is “Sensor B”, the process proceeds to S21206. If the radiation detecting device to use is “Sensor A”, the process proceeds to S21207.

In S21206, the control device 20 determines “Camera B” as the device to use and determines “Rotation 270 deg” and “Scale 50%” as the optical image geometry, on the basis of the table of radiography information management method (d).

In S21207, the control device 20 determines “Camera A” as the device to use and determines “Rotation 0 deg” and “Scale 100%” as the optical image geometry, on the basis of the table of radiography information management method (d).

### Effects

According to the eighth embodiment above, the devices to use and the optical image geometry can be determined by selecting the imaging type. Accordingly, an optical image from an appropriate optical image capture device can be displayed at an appropriate angle and magnification in the optical image display area 551 of the optical image window 550.

### Ninth Embodiment

In the sixth embodiment, the devices to use and the optical image geometry are determined by selecting the imaging type. The ninth embodiment describes a case of selecting multiple optical image capture devices and a case in which the optical image capture device to select is “N/A”. Note that the configuration for realizing the ninth embodiment is similar to that of the sixth embodiment except for the portion related to the abovementioned characteristic portion. Accordingly, the similar portions of the configuration are denoted and described using similar signs, and a detailed description of such portions is omitted.

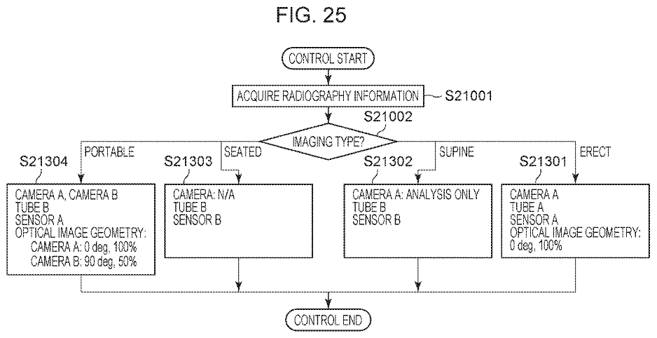

FIG. 25 is a diagram illustrating a flowchart of radiography information management method (e).

In S21001, the control device 20 acquires radiography information in a manner similar to the sixth embodiment.

In S21002, the control device 20 acquires the imaging type in a manner similar to the sixth embodiment. If the imaging type is “Erect”, the process proceeds to S21301. If the imaging type is “Supine”, the process proceeds to S21302. If the imaging type is “Seated”, the process proceeds to S21303. If the imaging type is “Portable”, the process proceeds to S21304.

In S21301, the control device 20 determines “Camera A”, “Tube A”, and “Sensor A” as the devices to use and determines “Rotation 0 deg” and “Scale 100%” as the optical image geometry, on the basis of the table of radiography information management method (e).

In S21302, the control device 20 determines “Camera A: Analysis only”, “Tube B”, and “Sensor B” as the devices to use, on the basis of the table of radiography information management method (e). Here, “Camera A: Analysis only” indicates that the device is only used for analysis and not used for optical image display. That is, only information based on an analysis result from the optical image analysis unit 205 is displayed in the optical image window 550. This is because, depending on the imaging type and the area of the body to be imaged, there may be cases where it is not desired to display the optical image due to privacy issues or the like, but it is desired to display the analysis result regarding the optical image.

In S21303, the control device 20 determines “Camera: N/A”, “Tube B”, and “Sensor B” as the devices to use, on the basis of the table of radiography information management method (e). Here, “Camera: N/A” means that an optical image capture device is not used. This is because, depending on the imaging type and the area of the body to be imaged, there may be cases where the radiography procedure and area of the body to be imaged are limited, and the optical image capture device is not used when confirmation using the optical image capture device is not essential.

In S21304, the control device 20 determines “Camera A, Camera B”, “Tube A”, and “Sensor A” as the devices to use and determines “Camera A: 0 deg, 100%” and “Camera B: 90 deg, 50%” as the optical image geometry, on the basis of the table of radiography information management method (e).

This assumes the use of various types of optical image capture devices, such as an optical image capture device mounted on the ceiling or the like. In the case of displaying optical image obtained from multiple optical image capture devices, the image to display by analyzing an acquired image may be selected automatically. For example, the optical image capture device at the closest distance to the subject or the largest image of the subject may be used. A configuration that uses the optical image from an optical image capture device with few obstructions between the optical image capture device and the subject is also possible. The optical images captured by multiple optical image capture devices may also be combined and displayed in the optical image display area 551 of the optical image window 550.

### Effects

According to the ninth embodiment above, the devices to use and the optical image geometry can be determined by selecting the imaging type. Accordingly, an optical image from an appropriate optical image capture device can be displayed at an appropriate angle and magnification in the optical image display area 551 of the optical image window 550.

### Other Examples

The present disclosure is not limited to the examples above. Various modifications (including organic combinations of the examples) are possible based on the gist of the disclosure, and such modifications are not excluded from the scope of the disclosure. That is, all configurations obtained by combining the examples described above and modifications thereof are also included in the present disclosure.

FIG. 1 illustrates an example of a system with two of each device, as with the radiation detecting device 10 (10A, 10B), the radiation generating device 30 (30A, 30B), and the optical image capture device 40 (40A, 40B), but there may also be three or more of each device.

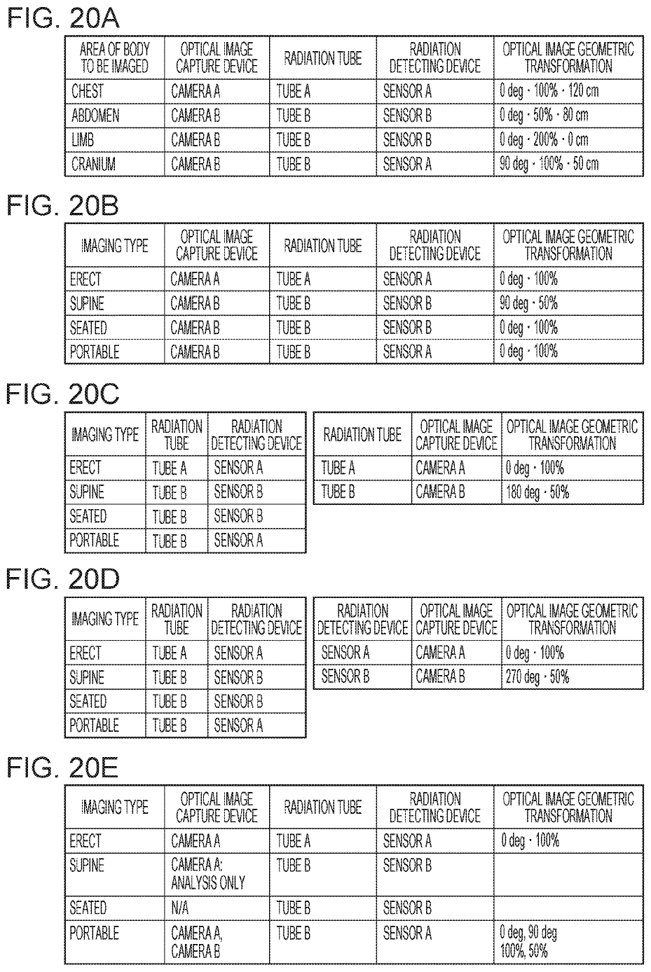

The information on the devices to use and the optical image geometry associated with the radiography information is not limited to the information given in FIGS. 20A to 20E. The radiography environment is expected to be a variety of environments. For example, it is assumed that in some cases, the optical image capture device may be mounted on the ceiling or the like of the radiography room, and not mounted close to the radiation tube. Accordingly, the user may also be able to edit the information on the devices to use and the optical image geometry associated with the radiography information such as the area of the body to be imaged and the imaging type. By determining specific information on the devices to use and the optical image geometry associated with the radiography information in advance, the user can later select radiography information to call up similar information on the devices to use and the optical image geometry.

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)?), a flash memory device, a memory card, and the like.

The processor or circuit may include a graphics processing unit (GPU) or a field-programmable gate array (FPGA). The processor or circuit may also include a digital signal processor (DSP), a data flow processor (DFP), or a neural processing unit (NPU).

The radiography system in each of the embodiments described above may be realized in the form of a standalone device or in the form of a combination of multiple devices capable of communicating with each other to execute the processing described above, and both of these forms are included in the embodiments of the present disclosure. A common server device or server cluster may also execute the processing described above. It is sufficient if the multiple devices constituting the radiography system are capable of communicating at a prescribed communicate rate, and the multiple devices do not need to be present with the same facility or in the same country.

The embodiments include an implementation in which a software program for realizing the functions of the embodiments described above is supplied to a system or device, and a computer of the system or device reads out and executes the code of the supplied program.

Consequently, the program code itself, which is installed in the computer to realize the processing pertaining to the embodiments on the computer, is also one of the embodiments of the present disclosure. Moreover, an OS or the like running on the computer may perform some or all of the actual processing on the basis of instructions included in the program read out by the computer, and the functions of the embodiments described above may also be realized by such processing.

### Appendices

The disclosure of the embodiments includes the following configurations and methods.

### Appendix 1

A radiographic imaging system including a radiation emitting device that emits radiation, a radiographic imaging device for acquiring a radiograph on the basis of radiation that has passed through a subject, an optical image capture device that captures an optical image of the subject, and an information processing device that enables information pertaining to radiography using the radiographic imaging device to be viewed on a display unit, the radiographic imaging system characterized in that
the information processing device controls the display of the optical image acquired from the optical image capture device in association with the progress of a predetermined step among a plurality of steps for performing the radiography.

### Appendix 2

The radiographic imaging system according to appendix 1, characterized in that the predetermined step is a step of instructing the radiographic imaging device to prepare for imaging.

### Appendix 3

The radiographic imaging system according to appendix 1 or 2, characterized in that the predetermined step is a step in which the radiographic imaging device starts an imaging operation.

### Appendix 4

The radiographic imaging system according to any one of appendices 1 to 3, characterized in that
the information processing device is capable of a superimposed display in which the radiograph acquired by the radiographic imaging and the optical image are superimposed onto one another, and
in the superimposed display, the optical image is displayed at a smaller size than the radiograph.

### Appendix 5

The radiographic imaging system according to any one of appendices 1 to 4, characterized in that a screen displayed by the information processing device includes a selection object for turning on or turning off the display of the optical image.

### Appendix 6

The radiographic imaging system according to any one of appendices 1 to 5, characterized in that
the information processing device executes predetermined processing as a process of displaying the optical image on the display unit, on the basis of information related to a status of the subject, and
the predetermined process is any of the following: displaying; not displaying; and performing image alteration and displaying an altered image.

### Appendix 7

The radiographic imaging system according to any one of appendices 1 to 5, characterized in that the information processing device displays, on the display unit, status information pertaining to the subject on the basis of an analysis result regarding the optical image.

### Appendix 8

The radiographic imaging system according to any one of appendices 1 to 5, characterized in that the information processing device displays the status information and the optical image on a same screen of the display unit.

### Appendix 9

The radiographic imaging system according to any one of appendices 1 to 5, characterized in that the information processing device displays the status information on a screen of the display unit without displaying the optical image.

### Appendix 10

The radiographic imaging system according to any one of appendices 1 to 7, characterized in that the information processing device saves the optical image in association with examination information pertaining to the radiography.

### Appendix 11

The radiographic imaging system according to appendix 10, characterized in that the information processing device determines to save the optical image, not save the optical image, or perform image alteration on the optical image and save an altered image, on the basis of information related to the status of the subject.

### Appendix 12

The radiographic imaging system according to appendix 6, characterized in that the image alteration is for mosaicing or replacing all or part of the optical image with figures or the like.

### Appendix 13

The radiographic imaging system according to appendix 11, characterized in that the image alteration is for mosaicing or replacing all or part of the optical image with figures or the like.

### Appendix 14

An information processing device to be used in a radiographic imaging system comprising a radiation emitting device that emits radiation, a radiographic imaging device for acquiring a radiograph on the basis of radiation that has passed through a subject, and an optical image capture device that captures an optical image of the subject, the information processing device characterized by including:
means for controlling the display of the optical image acquired from the optical image capture device in association with the progress of a predetermined step among a plurality of steps for performing the radiography.

### Appendix 15

A method of controlling an information processing device to be used in a radiographic imaging system comprising a radiation emitting device that emits radiation, a radiographic imaging device for acquiring a radiograph on the basis of radiation that has passed through a subject, and an optical image capture device that captures an optical image of the subject, the method of controlling characterized by including:
a step for controlling the display of the optical image acquired from the optical image capture device in association with the progress of a predetermined step among a plurality of steps for performing the radiography.

### Appendix 16

A program for causing a computer to execute the method of controlling according to appendix 15.

### Appendix 17

A radiographic imaging system including a radiation emitting device that emits radiation, a radiographic imaging device for acquiring a radiograph on the basis of radiation that has passed through a subject, an optical image capture device that captures an optical image of the subject, and an information processing device that enables information pertaining to radiography using the radiographic imaging device to be viewed on a display unit, the radiographic imaging system characterized in that
the information processing device includes:
   means for acquiring the optical image from the optical image capture device;
   means for acquiring information related to an imaging pose; and
   means for providing a notification of information related to the suitability of the imaging on the basis of the optical image and the information on the imaging pose.

### Appendix 18

An information processing device to be used in a radiographic imaging system including a radiation emitting device that emits radiation, a radiographic imaging device for acquiring a radiograph on the basis of radiation that has passed through a subject, and an optical image capture device that captures an optical image of the subject, the information processing device characterized by including:
means for acquiring the optical image from the optical image capture device;
means for acquiring information related to an imaging pose; and
means for providing a notification of information related to the suitability of the imaging on the basis of the optical image and the information on the imaging pose.

### Appendix 19

The information processing device according to appendix 18, characterized by including:
a display unit that displays information; and
means for causing the display unit to display information related to the suitability of the imaging.

### Appendix 20

The information processing device according to appendix 19, characterized by including means for causing the display unit to display an indication of whether or not a human body is detected from the optical image.

### Appendix 21

The information processing device according to appendix 20, characterized by including means for causing the display unit to superimpose onto the optical image a target area of consistency determination specified by the human body detection.

### Appendix 22

The information processing device according to any one of appendices 19 to 21, characterized by including: means for causing the display unit to display a notification indicating consistency when the position, type, and posture of an area of the human body obtained from the optical image is consistent with the information related to the imaging pose.

### Appendix 23

The information processing device according to any one of appendices 19 to 22, characterized by including means for causing the display unit to display a notification indicating inconsistency when information on any of the position, type, and posture of an area of the human body obtained from the optical image is not consistent with the information related to the imaging pose.

### Appendix 24

The information processing device according to any one of appendices 19 to 23, characterized by including means for causing the display unit to display an indication that determination is not possible when consistency with the information related to the imaging pose cannot be confirmed from information obtained from the optical image.

### Appendix 25

The information processing device according to appendix 18, characterized by including determining means for making a determination on the basis of information on the pose of the subject acquired from the optical image and the information on the imaging pose.

### Appendix 26

The information processing device according to appendix 25, characterized in that, when making a consistency determination, the determining means detects a human body to be determined.

### Appendix 27

The information processing device according to appendix 26, characterized in that the determining means determines consistency when the position, type, and posture of an area of the human body to be determined in the consistency determination match the information related to the imaging pose.

### Appendix 28

The information processing device according to appendix 26 or 27, characterized in that the determining means determines inconsistency when at least one of the position, type, and posture of an area of the human body to be determined in the consistency determination differs from the information related to the imaging pose.

### Appendix 29

The information processing device according to any one of appendices 26 to 28, characterized in that the determining means determines that determination is not possible when at least one of the position, type, and posture of an area of the human body to be determined in the consistency determination cannot be determined with regard to consistency.

### Appendix 30

The information processing device according to any one of appendices 26 to 29, characterized in that the determining means makes the consistency determination upon receiving input of the optical image with a decimated frame rate lower than an acquisition frame rate of the image acquiring means.

### Appendix 31

A method of controlling an information processing device to be used in a radiographic imaging system including a radiation emitting device that emits radiation, a radiographic imaging device for acquiring a radiograph on the basis of radiation that has passed through a subject, and an optical image capture device that captures an optical image of the subject, the method of controlling characterized by including:
a step for acquiring the optical image from the optical image capture device;
a step for acquiring information related to an imaging pose; and
a step for providing a notification of information related to the suitability of the imaging on the basis of the optical image and the information on the imaging pose.

### Appendix 32

A radiographic imaging system including a radiation emitting device that emits radiation, a radiographic imaging device for acquiring a radiograph on the basis of radiation that has passed through a subject, a plurality of optical image capture devices comprising at least a first optical image capture device and a second optical image capture device that each capture an optical image of the subject, and an information processing device that enables information pertaining to radiography using the radiographic imaging device to be viewed on a display unit, the radiographic imaging system characterized in that
the information processing device includes:
   means for acquiring radiography information about the next instance of radiography; and
   means for determining, on the basis of the radiography information, the optical image to be displayed on the display unit in a period until the next instance of radiography, from out of the optical images acquirable from the first optical image capture device and the second optical image capture device.

### Appendix 33

An information processing device to be used in a radiographic imaging system including a radiation emitting device that emits radiation, a radiographic imaging device for acquiring a radiograph on the basis of radiation that has passed through a subject, and a plurality of optical image capture devices comprising at least a first optical image capture device and a second optical image capture device that each capture an optical image of the subject, the information processing device characterized by including: a display unit that can display information;
means for acquiring radiography information related to the next instance of radiography; and
means for determining, on the basis of the radiography information, the optical image to be displayed on the display unit in a period until the next instance of radiography, from out of the optical images acquirable from the first optical image capture device and the second optical image capture device.

### Appendix 34

The information processing device according to appendix 33, characterized in that, in the period until the next instance of radiography, the information processing device acquires an optical image using only one of either the first optical image capture device or the second optical image capture device, and causes the display unit to display the acquired optical image.

### Appendix 35

The information processing device according to appendix 34, characterized in that the information processing device determines which optical image capture device to use in the acquisition of an optical image from among the plurality of optical image capture devices on the basis of a predetermined conditions, the predetermined condition including any one of the following: the distance between the subject and the optical image capture device is short; there are few obstructions between the subject and the optical image capture device; and the image of the subject is large.

### Appendix 36

The information processing device according to appendix 33, characterized in that, in the period until the next instance of radiography, the information processing device combines a first optical image acquired from the first optical image capture device with a second optical image from the second optical image capture device and causes the display unit to display the combined image.

### Appendix 37

The information processing device according to any one of appendices 33 to 36, characterized in that
the radiographic imaging system comprises a plurality of radiation emitting devices including the radiation emitting device, and
the information processing device includes means for setting, on the basis of the radiography information, one radiation emitting device from among the plurality of radiation emitting devices as a device to use.

### Appendix 38

The information processing device according to any one of appendices 33 to 37, characterized in that
the radiographic imaging system comprises a plurality of radiographic imaging devices including the radiographic imaging device, and
the information processing device sets, on the basis of the radiography information, one radiographic imaging device from among the plurality of radiographic imaging devices as a device to use.

### Appendix 39

The information processing device according to any one of appendices 33 to 38, characterized in that the information processing device includes means for controlling a screen to be displayed on the display unit on the basis of any one of the following pieces of information: the radiograph obtained from the radiographic imaging device; the optical image obtained from the optical image capture device; and an analysis result obtained by analyzing the optical image.

### Appendix 40

The information processing device according to any one of appendices 33 to 39, characterized in that the information processing device controls, on the basis of the radiography information, the turning-on and turning-off of the display of the optical image obtained from the optical image capture device.

### Appendix 41

The information processing device according to any one of appendices 33 to 40, characterized in that the information processing device controls, on the basis of the radiography information, the turning-on and turning-off of the display of an analysis result based on the optical image obtained from the optical image capture device.

### Appendix 42

The information processing device according to any one of appendices 33 to 41, characterized in that the information processing device performs image processing on the optical image acquired from the optical image capture device, the image processing being based on geometry information obtained on the basis of the radiography information.

### Appendix 43

The radiographic imaging system according to appendix 42, characterized in that the geometry information includes any one of the following: rotation information; enlargement or reduction information; and information indicating the center coordinates of the optical image.

### Appendix 44

The information processing device according to any one of appendices 33 to 43, characterized in that the radiography information includes any one of the following: an imaging procedure (erect, supine, seated, portable); an area of the body to be imaged, and patient information.

### Appendix 45

A method of controlling an information processing device to be used in a radiographic imaging system including a radiation emitting device that emits radiation, a radiographic imaging device for acquiring a radiograph on the basis of radiation that has passed through a subject, and a plurality of optical image capture devices comprising at least a first optical image capture device and a second optical image capture device that each capture an optical image of the subject, the method of controlling characterized by including:
a step for acquiring radiography information related to the next instance of radiography; and
a step for determining, on the basis of the radiography information, the optical image to be displayed on a display unit in a period until the next instance of radiography, from out of the optical images acquirable from the first optical image capture device and the second optical image capture device.

### Appendix 46

A program causing a computer to execute the method of controlling according to appendix 45.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

What is claimed is:

1. An information processing apparatus for use in a radiographic imaging system, the information processing apparatus comprising:
one or more controllers configured to:
cause a display unit to display a screen including a first display area in which a radiographic image acquired by radiography is displayable; and
cause the display unit to display, in a manner superimposed on the first display area, a second display area including an optical image of a subject of the radiography and determination-result information corresponding to a result of determination based on the optical image.

2. The information processing apparatus according to claim 1,
wherein the second display area includes:
an optical image display area in which the optical image is displayed; and
a notification area in which the determination-result information is displayed.

3. The information processing apparatus according to claim 1,
wherein the result of determination based on the optical image includes a detection processing result of the subject based on the optical image.

4. The information processing apparatus according to claim 1,
wherein the determination-result information includes information relating to suitability of the radiography.

5. The information processing apparatus according to claim 1,
wherein the determination-result information includes information indicating whether the subject is detected from the optical image.

6. The information processing apparatus according to claim 1,
wherein the one or more controllers are further configured to acquire examination information relating to the radiography, and
wherein the result of determination includes a result of determining whether information obtained from the optical image is consistent with the examination information or information relating to an imaging pose of the radiography.

7. The information processing apparatus according to claim 6,
wherein the information obtained from the optical image includes at least one of a body part, a direction, or laterality of the subject.

8. The information processing apparatus according to claim 1,
wherein the information processing apparatus is included in a radiographic imaging system comprising:
a radiographic imaging device configured to acquire the radiographic image by radiography; and
an optical image capture device configured to optically capture the subject of the radiography to acquire the optical image.

9. An information processing apparatus for use in a radiographic imaging system, the information processing apparatus comprising:
one or more controllers configured to cause a display unit to display suitability information indicating whether radiography setting information for radiography is suitable for determining a pose of a subject of the radiography based on an optical image of the subject.

10. The information processing apparatus according to claim 9,
wherein the radiography setting information is determined to be unsuitable for determining the pose of the subject based on the optical image when the radiography setting information does not specify a body part to be imaged.

11. The information processing apparatus according to claim 9,
wherein the radiography setting information is determined to be unsuitable for determining the pose of the subject based on the optical image when the radiography setting information specifies a body part that cannot be analyzed based on the optical image.

12. The information processing apparatus according to claim 9,
wherein, when the radiography setting information is suitable for determining the pose of the subject based on the optical image, the one or more controllers are further configured to determine the pose of the subject based on the optical image and the radiography setting information.

13. The information processing apparatus according to claim 9,
wherein the one or more controllers are further configured to display, on a setting screen, a setting item corresponding to the radiography setting information in a manner distinguishable according to whether the radiography setting information is suitable or unsuitable for determining the pose of the subject based on the optical image.

14. The information processing apparatus according to claim 9,
wherein the information processing apparatus is included in a radiographic imaging system comprising:
a radiographic imaging device configured to acquire a radiographic image by radiography based on the radiography setting information; and
an optical image capture device configured to optically capture the subject of the radiography to acquire the optical image.

15. An information processing apparatus for use in a radiographic imaging system, the information processing apparatus comprising:
one or more controllers configured to:
acquire an optical image of a subject of radiography;
perform pose determination of the subject based on the optical image; and
cause a display unit to display in-progress information indicating that processing of the pose determination is being performed while the processing of the pose determination is being performed.

16. The information processing apparatus according to claim 15,
wherein the in-progress information includes an icon indicating that analysis of the optical image is being executed.

17. The information processing apparatus according to claim 16,
wherein the one or more controllers are configured to hide or gray out the icon when the processing of the pose determination is not being performed.

18. The information processing apparatus according to claim 15,
wherein the one or more controllers are further configured to cause the display unit to display determination-result information corresponding to a result of the pose determination after completion of the processing of the pose determination.

19. The information processing apparatus according to claim 18,
wherein the in-progress information and the determination-result information are displayed in a distinguishable manner.

20. The information processing apparatus according to claim 15,
wherein the information processing apparatus is included in a radiographic imaging system comprising:
a radiographic imaging device configured to acquire a radiographic image by radiography; and
an optical image capture device configured to optically capture the subject of the radiography to acquire the optical image.
